(19) United States
(12) Patent Application Publication
(10) Pub. No.: US 2008/0255545 A1
(43) Pub. Date: Oct. 16, 2008

(54) APPARATUS AND METHOD FOR TREATING THE INSIDE OF AN EYE

(76) Inventors: John M. Mansfield, Berlin, MA (US); Jon T. McIntyre, Newton, MA (US)

Correspondence Address: COOLEY GODWARD KRONISH LLP;ATTN: PATENT GROUP, Suite 1100, 777 - 6th Street, NW, WASHINGTON, DC, 20001, US

(21) Appl. No.: 11/733,273

(22) Filed: Apr. 10, 2007

(51) Int. Cl. A61B 18/24

(52) U.S. Cl. 606/4

(57) ABSTRACT

In one embodiment, an apparatus includes a first body having a proximal end and a distal end and defining a lumen, and a second body defining a second lumen. The first body is disposed within the lumen of the second body. The lumen of the first body is configured to receive a medical device for performing a medical procedure within at least a portion of an eye. The second body includes a sharpened distal end configured to penetrate a surface of the eye. A plurality of illumination fibers is disposed between the first body and the second body. In some embodiments, the lumen of the first body and the lumen of the second body are coaxial. In some embodiments, the sharpened distal end includes an angled surface.

### FIELD OF THE INVENTION

The disclosed invention relates generally to a medical device and more particularly to a medical device configured for use in the treatment of the inside of an eye.

### BACKGROUND

Various devices exists for performing surgical procedures inside an eye, such as endoscopic cyclophotocoagulation (ECP) and endoscopic vitrectomy. Such devices include endoscopes and probes that can provide illumination within an eye and/or provide laser energy. Known devices are used to treat various eye disorders, such as, for example, Age Related Macular Degeneration (AMD), which is a disease that causes progressive damage to the macula. Current treatments for AMD include the use of vitamin supplements, laser treatment (e.g., thermal or photodynamic therapy), and therapeutic agents or drugs. During photodynamic therapy, a drug is injected into a patient's arm intravenously, and then the drug is activated by shining a laser light into the patient's eye.

Diabetic Retinopathy (DR) is another disorder that can be treated with laser energy. DR is an eye disorder in which diabetes damages the small blood vessels in the retina. Laser treatment is the typical treatment for DR, or surgical removal of the vitreous gel (i.e., a vitrectomy). Laser treatment is also used to treat Glaucoma, which is a progressive optic neuropathy often associated with fluid pressure build up inside the eye.

For treatment of all of the above described eye disorders it is desirable to gain access to the inside of the eye to perform the particular procedure directly at the affected site within the eye. It is also desirable to provide illumination within the eye during the treatment. The devices and methods described herein provide the ability to penetrate the surface of the eye to directly treat the interior of the eye, provide illumination during treatment, and access to the interior of the eye for a medical tool or instrument to perform the particular procedure within the eye.

Thus, there is a need for an apparatus and method for treatment within an interior of an eye that provides penetration of the eye through a minimized puncture (avoiding larger incisions), access for other tools or instruments and illumination during the treatment.

### SUMMARY OF THE INVENTION

In one embodiment, an apparatus includes a first body having a proximal end and a distal end and defining a lumen, and a second body defining a second lumen. The first body is disposed within the lumen of the second body. The lumen of the first body is configured to receive a medical device for performing a medical procedure within at least a portion of an eye. The second body includes a sharpened distal end configured for tissue penetration into the eye. A plurality of illumination fibers are disposed between the first body and the second body. In some embodiments, the lumen of the first body and the lumen of the second body are coextensive. In some embodiments, the sharpened distal end includes an angled surface configured to facilitate insertion into an interior of the eye.

### BRIEF DESCRIPTION OF THE DRAWINGS

FIG. 1 is a schematic illustration of a medical device according to an embodiment of the invention.

FIG. 2 is a side view of a portion of a medical device according to an embodiment of the invention.

FIG. 3 is a distal end view of the medical device of FIG. 2 taken along line 3-3 in FIG. 2.

FIG. 4 is a side cross-sectional view of a distal portion of the medical device of FIG. 2 shown with a medical instrument inserted therethrough.

FIG. 5 is a side view shown partially in cross-section of a proximal portion of the medical device of FIG. 2.

FIG. 6 is a proximal end view of the medical device of FIG. 2 taken along line 6-6 in FIG. 2.

FIG. 7 is a side view shown partially in cross-section of a proximal portion of a medical device according to another embodiment of the invention.

FIG. 8 is a flowchart illustrating a method according to an embodiment of the invention.

### DETAILED DESCRIPTION

The apparatuses and methods described herein can be used for penetrating, accessing, and illuminating an eye. The apparatuses described herein include a sharpened distal end or tip that can be used to penetrate the outer layers of the eye resulting in a minimized entry site. A working channel is provided for inserting various medical devices through the apparatus to perform various medical procedures. Illumination fibers are also provided to provide illumination of the inside of the eye during treatment of the eye.

An apparatus as described herein can be used with various medical instruments that can be inserted through the working channel. For example, devices for administering drug therapies to the interior of eye can be inserted through the working channel and positioned at a desired treatment site within the eye. Such drug therapies may be desired, for example, in the treatment of dry or wet Age Related Macular Degeneration or Diabetic Retinopathy. Other devices, such as devices that provide laser energy or laser light, as with photodynamic therapy (PDT), or devices used in procedures for treating Glaucoma, such as probes (for cyclocryotherapy or cyclodiathermy), knives (for goniotomy) or shunt delivery systems, can also be used in conjunction with the medical devices described herein. The medical devices described herein may also be used in performing innovative procedures related to treating Glaucoma such as surgical procedures related to the ganglion cells, the optic neuro-network and/or the optic nerve. In some embodiments, a medical device as described herein is used to provide a working channel to treat intra ocular pressure concerns. Such therapies may include, for example, surgical procedures related to the ciliary process, schlemm's canal, trabecular meshwork or insertion of drug delivery devices.

In yet another embodiment, an apparatus is provided herein for use in the delivery of an implant to a desired location within the eye. For example, an implant, such as a neurostimulator or various microchips, for use in reestablishing vision can be delivered through the working channel to the back of the eye. Such a delivery system for implants is less invasive than known implantation procedures. In another example, an apparatus can be used in conjunction with a microscope positioned exterior to the eye that can be used to view through the lens of an eye. For example, illumination fibers within the apparatus can be used to provide lighting for viewing through the lens of the eye with the microscope.

In one embodiment, an apparatus includes a first body having a proximal end and a distal end and defining a lumen, and a second body defining a second lumen. The first body is disposed within the lumen of the second body. The lumen of the first body is configured to receive a medical device for performing a medical procedure within at least a portion of an eye. The second body includes a sharpened distal end configured to penetrate the eye. A plurality of illumination fibers are disposed between the first body and the second body. In some embodiments, the lumen of the first body and the lumen of the second body are coextensive. In some embodiments, the sharpened distal end includes an angled surface configured to facilitate insertion into the eye with minimal trauma.

In another embodiment, an apparatus includes a first elongate body and a second elongate body. The first elongate body has a proximal end and a distal end and defines a lumen. The lumen of the first elongate body is configured to receive a medical device to perform a procedure on a portion of an eye. The first elongate body is disposed within a lumen of the second elongate body, and the lumen of the first elongate body is coaxial with the lumen of the second elongate body. The second elongate body is configured to be inserted into a portion of an eye. A plurality of illumination members is disposed between the first elongate body and the second elongate body.

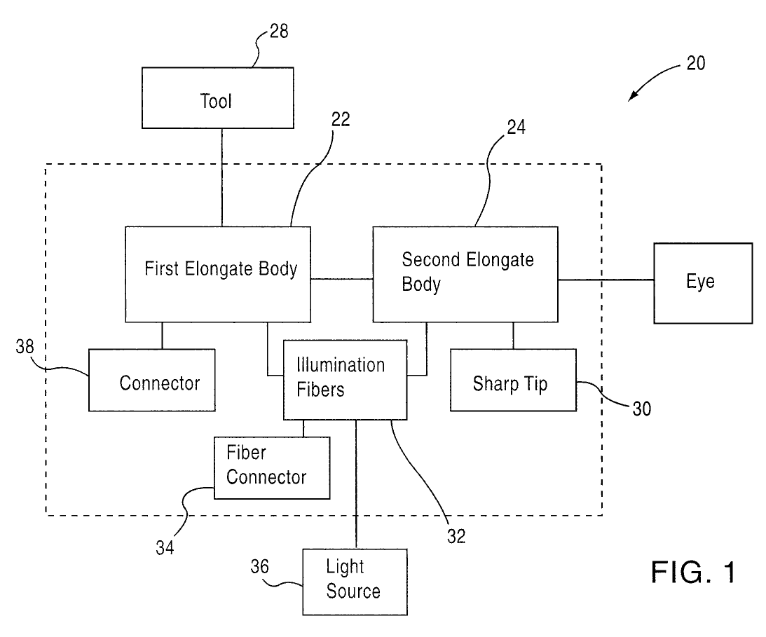

FIG. 1 is a schematic illustration of a medical device (also referred to herein as an “apparatus”) according to an embodiment of the invention. A medical device 20 includes a first elongate body 22 and a second elongate body 24. The first elongate body 22 can be disposed within a lumen (not shown in FIG. 1) of the second elongate body 24. The first elongate body 22 and the second elongate body 24 can be formed as two separate components coupled together, or alternatively can be formed as a single component. For example, the first elongate body 22 can be a first tubular member and the second elongate body 24 can be a second tubular member. The first elongate body 22 can then be inserted into a lumen of the second elongate body 24. In some embodiments, the first elongate body 22 and the second elongate body 24 share a common longitudinal axis such that the lumen of the first elongate body 22 is coaxial with the lumen of the second elongate body 24. In some embodiments, the second elongate body 24 can have, for example, an outer diameter of approximately 0.039 inches (0.99 mm), and a wall thickness of approximately 0.002 inches (0.05 mm); the first elongate body 22 can have, for example, an outer diameter of 0.0245 inches (0.62 mm), and a wall thickness of 0.001 inches (0.025 mm).

The first elongate body 22 also defines a lumen (not shown in FIG. 1) that can be used as a working channel for a medical instrument or tool 28 to be inserted therethrough and disposed at a desired treatment site within an eye. For example, a small needle (e.g., 25 gauge) can be inserted through the lumen of the first elongate body 22 and used to deliver drugs to the interior of an eye. In some embodiments, a laser fiber accessory can be inserted through the working channel of the first elongate body 22 and used for ablative or photo reactive therapies delivered to the inside of the eye. Thus, the medical device 20 can provide micro-access (e.g., <1 mm) through the working channel of the first elongate body 22 to a desired location within an interior of an eye.

The medical device 20 can also include a connector 38, such as a luer lock fitting coupled to a proximal end of the first elongate body 22. The connector 38 can include a seal, such as a passive seal that allows passage of a medical device without allowing fluid to pass through. In some embodiments, a medical device does not include a connector 38, but includes a seal coupled to the proximal end of the first elongate body 22. A seal can be formed with a flexible material, such as, for example, silicon or latex, and define a slit or opening to permit the passage of medical instrument(s) therethrough. To minimize or eliminate reaction issues, a seal can be formed with a flexible material, such as an organic, carbon based compound, or an inorganic, silicon based compound. The flexibility of the seal will allow the seal to conform to the surface of the medical instrument to prevent fluid from passing back through the seal.

The second elongate body 24 has a distal end that can be used to penetrate an outer surface of an eye. For example, the second elongate body 24 can include a sharpened distal tip 30 that can make a minimal puncture entry through the surface of an eye. The portion of the distal end of the second elongate body 24 that can be inserted into the eye can be, for example, approximately 4 cm. In some embodiments, a lubricous coating can be disposed on an outer surface of the second elongate body 24 to provide easier insertion into the eye. In some embodiments, anti-microbial coatings can also be disposed on an outer surface of the second elongate body 24.

The medical device 20 can also include one or more illumination fibers 32 that can be disposed in the lumen of the second elongate body 24 between the first elongate body 22 and the second elongate body 24, forming, for example, a ring configuration (e.g., illumination fibers 132 shown in FIG. 3). In some embodiments, the illumination fibers 32 are plastic optical fibers approximately 100 microns in diameter. The illumination fibers 32 can be coupled to an outer surface of the first elongate body 22 and/or to an inner surface of the second elongate body 24 with, for example, an adhesive. The illumination fibers 32 can extend from a proximal end of either the first elongate body 22 or the second elongate body 24 or both, and terminate at a distal end of either the first elongate body 22 and/or the second elongate body 24. The illumination fibers 32 provide illumination of an interior of an eye during a medical procedure performed on the eye.

In some embodiments, the illumination fibers 32 exit a proximal end of the second elongate body 24 and wrap around to one side of the first elongate body 22 and form into a bundle that terminates into a fiber optic connector 34. For example, the illumination fibers 32 can bifurcate into two bundles that then converge back into a single bundle (e.g., illumination fibers 132 shown in FIG. 6). A proximal end of the illumination fibers 32 can then be coupled to a single light source 36.

The light source 36 can provide, for example, white light that can be carried along the fibers 32 to a distal end of the medical device to illuminate the inside of the eye during a medical procedure. In some embodiments, one or more specific bandwidths of light can be provided through the illumination fibers 32. These bandwidths of light can be used, for example, to highlight different pathologies inside the eye. In some embodiments, the illumination fibers 32 can be divided to terminate at a proximal end of the second elongate body 24 into separate bundles each with an individual optical connector 34. This would enable the convenient and efficient delivery of multiple different bandwidths of light. Thus, illumination can be provided with white light or individual bandwidths of light, which can include one or more bandwidths anywhere along the spectrum of light. For example, narrow bandwidths that may be used include 385-415 nm, 415-445 nm, 470-500 nm, 500-530 nm, 520-540 nm and 580-620 nm. Different wavelength ranges can be used, for example, to detect veins vs. arteries, thick vs. small vessels, or deep vessels vs. superficial. Broad bandwidths can also be used that may overlap more than one of the narrow-bands previously mentioned.

In use, the sharpened distal end (e.g., distal tip 30) of the second elongate body 24 can be used to make a tiny puncture through an outer surface of an eye and a distal portion of the medical device 20 can be positioned at a desired treatment site in the interior of the eye. A second medical device, such as medical tool 28, can then be inserted through the lumen of the first elongate body 22 and used to perform a procedure in the inside of the eye. The illumination fibers 32 can provide illumination during the procedure. Some procedures can be performed by hand under direct viewing through, for example, a scope. In some cases, the medical device 20 can be manipulated during a procedure to ensure the medical device 20 is positioned in the correct or desired location. In some embodiments, a stabilizing device can be used to help maintain the position of the medical device 20. For example, in a procedure in which the medical device 20 is positioned at the back of an eye or near the optic nerve the use of a stabilizing structure may be desired.

Having described above various general principles, several exemplary embodiments of these concepts are now described. These embodiments are only examples, and many other configurations of medical device 20 and its various components are contemplated, and will be apparent to the artisan in view of the general principles described above and the exemplary embodiments.

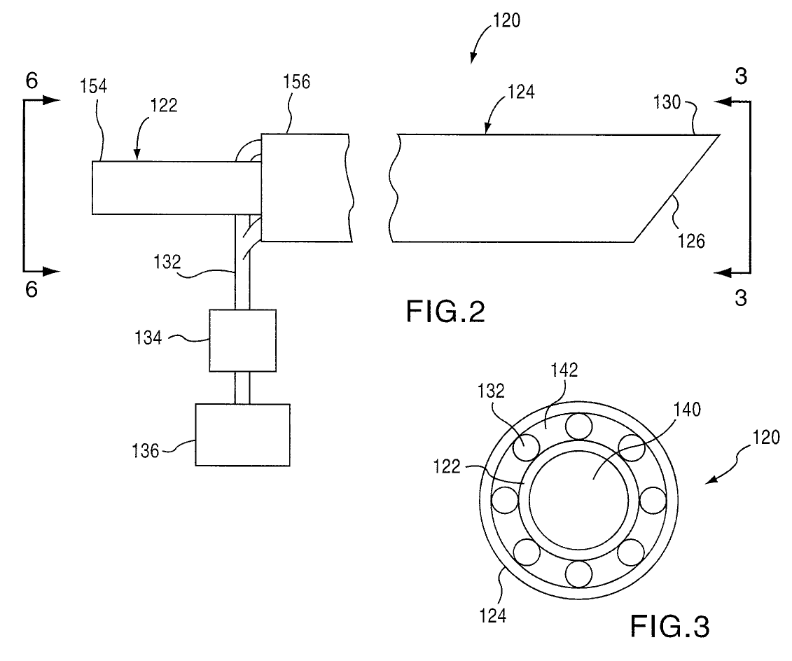

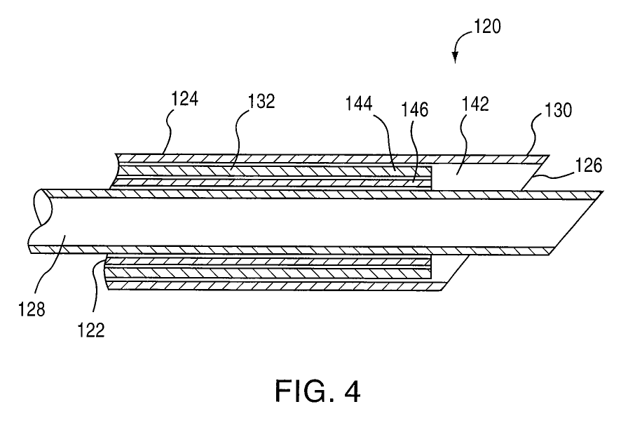

FIGS. 2-6 illustrate a medical device according to an embodiment of the invention. A medical device 120 includes a first elongate body 122 and a second elongate body 124. The first elongate body 122 defines a lumen 140 and is disposed within a lumen 142 defined by the second elongate body 124. The first elongate body 122 and the second elongate body 124 define a common longitudinal axis such that the lumen 140 and the lumen 142 are coaxial. In this embodiment, the second elongate body 124 has a sharpened distal tip 130 that defines an angled surface 126. The distal tip 130 is configured to penetrate through an outer surface of an eye. The lumen 140 of the first elongate body 122 is configured to receive a medical instrument, such as a needle 128, as shown in the cross-sectional view of FIG. 4. In this embodiment, a proximal end 154 of the first elongate body 122 extends beyond a proximal end 156 of the second elongate body 124.

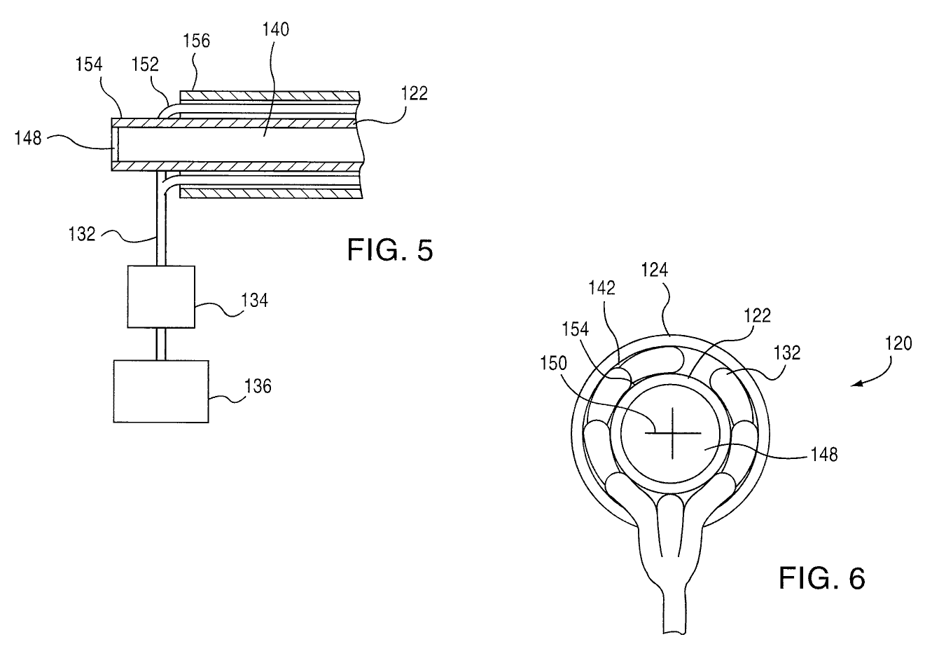

As shown in FIGS. 5 and 6, a seal 148 is disposed at the proximal end 154 of the first elongate body 122. The seal 148 includes a slit or aperture 150 (see FIG. 6) to allow for the passage of a medical instrument through the lumen 140 of the first elongate body 122, while not permitting the passage of fluid back through the seal 148, for example, fluid from the eye that has become disposed within the lumen 140 of the first elongate body 122.

The medical device 120 also includes multiple illumination fibers 132 disposed within the lumen 142 of the second elongate body 124 between the first elongate body 122 and the second elongate body 124. As shown in FIG. 4, a distal end 144 of the illumination fibers 132 terminate at a distal end 146 of the first elongate body 122. In other embodiments, the distal end 144 of the illumination fibers can terminate at a different location. For example, the illumination fibers can terminate at a distal end of the second elongate body 124. As shown in FIG. 5, a proximal end 152 of the illumination fibers 132 are wrapped to the sides of the first elongate body 122 and bundled into a single optical connector 134. By wrapping or bundling the fibers 132 into a single connector 134, the illumination fibers 132 can be coupled to a single light source 136.

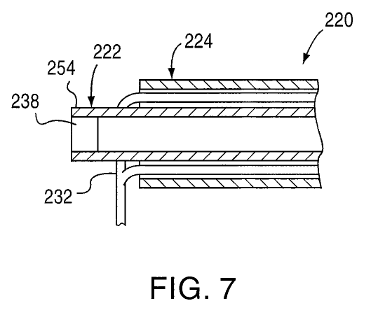

FIG. 7 illustrates a portion of an alternative embodiment of a medical device. A medical device 220 includes a first elongate body 222 disposed within a second elongate body 224 and multiple illumination fibers 232 disposed between the first elongate body 222 and the second elongate body 224. Medical device 220 is similar to the previous embodiment, except medical device 220 includes a connector 238 disposed at a proximal end 254 of the first elongate body 222. The connector 238 can be, for example, a luer lock fitting and can optionally include a seal similar to seal 148 of medical device 120.

The components of a medical device 20, 120, 220 can be formed with various materials. For example, the first elongate body and the second elongate body can each be formed with materials suitable for medical use, such as polyimide, polyamide, polycarbonate, stainless steel and others. Similarly, the illumination fibers can be formed with, for example, a polymethyl-methacrylate (PMMA) core and a fluorinated polymer cladding.

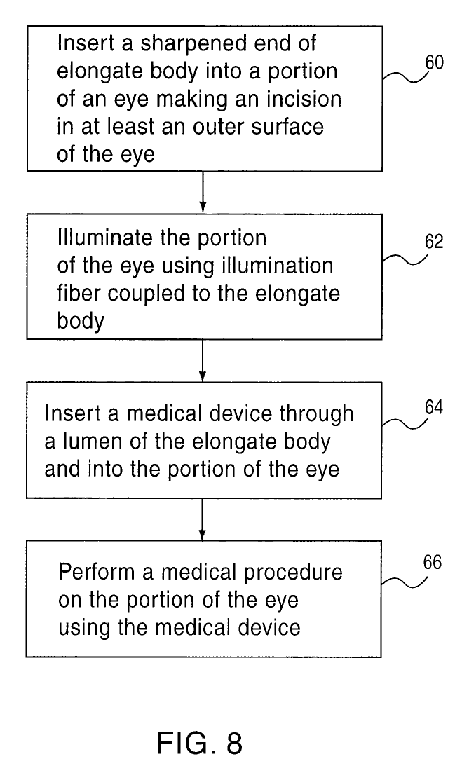

FIG. 8 illustrates a method according to an embodiment of the invention. A method includes at 60, inserting a sharpened distal end of an elongate body into a portion of an eye such that the distal end penetrates a surface of the eye. At 62, the portion of the eye is illuminated using an illumination fiber coupled to the elongate body. A medical device, such as a small gauge needle, an endoscope or laser is inserted through a lumen of the elongate body and into the eye at 64. At 66, a medical procedure is performed on the eye using the medical device. In some embodiments, the medical procedure performed includes delivering a therapeutic agent to the portion of the eye. In other embodiments, the medical procedure includes providing laser energy to a targeted location within the eye. In still other embodiments, the method includes insertion of an implant to a location within the eye.

### Conclusion

While various embodiments of the invention have been described above, it should be understood that they have been presented by way of example only, and not limitation. Thus, the breadth and scope of the invention should not be limited by any of the above-described embodiments, but should be defined only in accordance with the following claims and their equivalents.

The previous description of the embodiments is provided to enable any person skilled in the art to make or use the invention. While the invention has been particularly shown and described with reference to embodiments thereof, it will be understood by those skilled in art that various changes in form and details may be made therein without departing from the spirit and scope of the invention.

For example, a medical device 20, 120, 220 can include any combination or sub-combination of the various features and components described herein. Thus, a medical device 20, 120, 220 can include other configurations, shapes and materials not specifically illustrated, while still remaining within the scope of the invention. For example, the quantity of illumination fibers, as well as the configuration or arrangement of the illumination fibers can vary. Thus, in some embodiments, a single illumination fiber can be used. In addition, the sharp distal end of the second elongate body can have other shapes, sizes and or configurations.

What is claimed is:

1. An apparatus, comprising:
a first elongate body having a proximal end and a distal end and defining a lumen;
a second elongate body defining a second lumen, the first elongate body being disposed within the lumen of the second elongate body,
the lumen of the first elongate body configured to receive a medical device for performing a medical procedure within at least a portion of an eye, the second elongate body including a sharpened distal end configured to penetrate the eye; and
a plurality of illumination fibers disposed between the first elongate body and the second elongate body.

2. The apparatus of claim 1, wherein the sharpened distal end includes an angled surface configured to penetrate an outer layer of the eye.

3. The apparatus of claim 1, wherein the plurality of illumination fibers are formed in an annular ring and terminate at the distal end of the first elongate body.

4. The apparatus of claim 1, further comprising:
a connector, the proximal end of the first elongate body coupled to the connector, the connector including a seal configured to permit the insertion of the medical device into the lumen of the first elongate body, and further configured to prevent fluid from passing through the seal when the medical device is inserted.

5. The apparatus of claim 1, wherein the illumination fibers are coupled to a single fiber-optic connector.

6. The apparatus of claim 1, wherein the illumination fibers are coupled to multiple fiber-optic connectors.

7. The apparatus of claim 1, wherein the first elongate body is fixedly coupled to the second elongate body.

8. The apparatus of claim 1, wherein the lumen of the first elongate body is coaxial with the lumen of the second elongate body.

9. The apparatus of claim 1, further comprising:
a lubricous coating disposed on an outer surface of the second elongate body.

10. The apparatus of claim 1, further comprising:
an anti-microbial coating disposed on an outer surface of the second elongate body.

11. The apparatus of claim 1, wherein the plurality of illumination fibers are illuminated with a bandwidth of light of one of 385-415 nm, 415-445 nm, 470-500 nm, 500-530 nm, 520-540 nm and 580-620 nm.

12. An apparatus, comprising:
a first elongate body having a proximal end and a distal end and defining a lumen;
a second elongate body defining a lumen, the first elongate body disposed within the lumen of the second elongate body, the lumen of the first elongate body being coaxial with the lumen of the second elongate body, the second elongate body configured to be inserted into a portion of an eye; and
a plurality of illumination fibers disposed between the first elongate body and the second elongate body, the lumen of the first elongate body configured to receive a medical device to perform a procedure on a portion of the eye.

13. The apparatus of claim 12, wherein the second elongate body includes a sharpened distal end configured to penetrate an outer layer of the eye.

14. The apparatus of claim 12, wherein the second elongate body includes a distal end having an angled surface configured to penetrate into the eye.

15. The apparatus of claim 12, wherein the plurality of illumination fibers are formed in an annular ring and terminate at the distal end of the first elongate body.

16. The apparatus of claim 12, further comprising:
a connector, the proximal end of the first elongate body coupled to the connector, the connector including a seal configured to permit the insertion of the medical device into the lumen of the first elongate body, and further configured to prevent fluid from passing through the seal when the medical device is inserted.

17. The apparatus of claim 12, wherein the illumination fibers are coupled to a single fiber optic connector.

18. The apparatus of claim 12, wherein the first elongate body is fixedly coupled to the second elongate body.

19. The apparatus of claim 12, wherein the illumination fibers are illuminated with multiple bandwidths of light.

20. A method, comprising:
inserting a sharpened distal end of an elongate body into a portion of an eye such that the distal end makes an incision in at least an outer surface of the eye;
illuminating the portion of the eye using an illumination fiber coupled to the elongate body; and
inserting a medical device through a lumen defined by the elongate body and into the portion of the eye to perform a medical procedure on the portion of the eye.

21. The method of claim 20, further comprising:
performing a medical procedure on the portion of the eye using the medical device.

22. The method of claim 20, wherein the inserting the medical device includes inserting a device to deliver a therapeutic agent to the portion of the eye.

23. The method of claim 20, wherein the inserting the medical device includes inserting a device to provide laser energy to the portion of the eye.

24. The method of claim 20, wherein in the elongate body includes a first body defining a lumen and a second body defining a lumen, the lumen of the first body being coaxial with the lumen of the second body.
